(19) United States
(12) Patent Application Publication
(10) Pub. No.: US 2012/0209121 A1
(43) Pub. Date: Aug. 16, 2012

(54) ULTRASOUND PROBE INCLUDING A SECURING MEMBER

(75) Inventor: AURELIE BOUDIER, BUC (FR)

(73) Assignee: GENERAL ELECTRIC COMPANY, SCHENECTADY, NY (US)

(21) Appl. No.: 13/028,013

(22) Filed: Feb. 15, 2011

(51) Int. Cl. A61B 8/00

(52) U.S. Cl. 600/459

(57) ABSTRACT

An ultrasound probe includes a proximal portion including a transducer array, a distal portion, and a connecting portion configured to couple the scanning portion to the distal portion, the connecting portion sized to be received between two fingers to enable the ultrasound probe to be secured to an operator's palm. An imaging system including the ultrasound probe is also described herein.

### BACKGROUND OF THE INVENTION

The subject matter described herein generally relates to ultrasound probes, and more particularly to a device for securing an ultrasound probe to a user's hand.

Medical imaging systems, and in particular, ultrasound systems typically include ultrasound scanning devices, such as, ultrasound probes having different control components and transducers that allow for performing various different ultrasound scans (e.g., different imaging of a volume or body). These ultrasound probes may include control components within different portions of the probe, including, for example, the probe handle. These control components within the probe allow for controlling operation of the probe by an ultrasound system, for example, to operate in different modes, such as, amplitude mode (A-mode), brightness mode (B-1 mode), power Doppler mode, color imaging mode, among others.

Ultrasound examinations are non-invasive and may be performed concurrently with other examinations or procedures. For example, ultrasound examinations may be performed concurrently with a rheumatology procedure, an anesthesia procedure, and/or a neonatology procedure. When the ultrasound examination is performed concurrently with other procedures, the operator typically utilizes one hand to hold and operate the ultrasound probe and the other hand to operate equipment associated with the other procedure. However, for many procedures performed concurrently with the ultrasound examination, the operator may desire to utilize both hands. For example, it may be difficult for a single operator to insert a catheter into the patient while concurrently observing the location of the catheter using the ultrasound probe. In this case, the operator may simply choose to set aside the ultrasound probe and utilize both hands to insert the catheter or utilize a second operator to assist in either operating the ultrasound probe or inserting the catheter. In other cases, the operator may desire to set aside the ultrasound probe to operate various controls or devices associated with the other procedures. Therefore, conventional probe arrangements limit the use of these probes during concurrent examinations or procedures.

### SUMMARY OF THE INVENTION

In one embodiment, an ultrasound probe is provided. The ultrasound probe includes a proximal portion including a transducer array, a distal portion, and a connecting portion configured to couple the scanning portion to the distal portion, the connecting portion sized to be received between two fingers to enable the ultrasound probe to be secured to an operator's palm.

In another embodiment, another ultrasound probe is provided. The ultrasound probe includes a housing having a proximal end and a distal end, the distal end being curved to conform to an operator' palm, a transducer array located within the housing, and a strap coupled to the housing, the strap configured to at least partially circumscribe the hand and to secure the ultrasound probe to the operator's palm.

In a further embodiment, an imaging system is provided. The imaging system includes an ultrasound probe. The ultrasound probe includes a proximal portion including a transducer array, a distal portion, and a connecting portion configured to couple the scanning portion to the distal portion, the connecting portion sized to be received between two fingers to enable the ultrasound probe to be secured to an operator's palm. The ultrasound imaging system also includes a processor communicatively coupled with the ultrasound probe, the processor receiving ultrasound data from the ultrasound probe and generating at least one images based on the ultrasound data.

### BRIEF DESCRIPTION OF THE DRAWINGS

FIG. 1 is a simplified block diagram of an exemplary ultrasound probe formed in accordance with various embodiments.

FIG. 2 is a simplified block diagram of another exemplary ultrasound probe formed in accordance with various embodiments.

FIG. 3 is a perspective view of the ultrasound probe shown in FIG. 1.

FIG. 4 is a perspective view of the ultrasound probe shown in FIG. 1 in a first operational position.

FIG. 5 is another perspective view of the ultrasound probe shown in FIG. 1 in a second operational position.

FIG. 6 is a perspective view of another ultrasound probe formed in accordance with various embodiments.

FIG. 7 is a perspective view of the ultrasound probe shown in FIG. 6 in an exemplary operational position.

FIG. 8 is a perspective view of another ultrasound probe formed in accordance with various embodiments.

FIG. 9 is a perspective view of an ultrasound glove formed in accordance with various embodiments.

FIG. 10 illustrates a block diagram of an exemplary ultrasound system that is formed in accordance with various embodiments.

### DETAILED DESCRIPTION OF THE DRAWINGS

The foregoing summary, as well as the following detailed description of certain embodiments of the present invention, will be better understood when read in conjunction with the appended drawings. The figures illustrate diagrams of the functional blocks of various embodiments. The functional blocks are not necessarily indicative of the division between hardware circuitry. Thus, for example, one or more of the functional blocks (e.g., processors or memories) may be implemented in a single piece of hardware (e.g., a general purpose signal processor or a block or random access memory, hard disk, or the like). Similarly, the programs may be stand alone programs, may be incorporated as subroutines in an operating system, may be functions in an installed imaging software package, and the like. It should be understood that the various embodiments are not limited to the arrangements and instrumentality shown in the drawings.

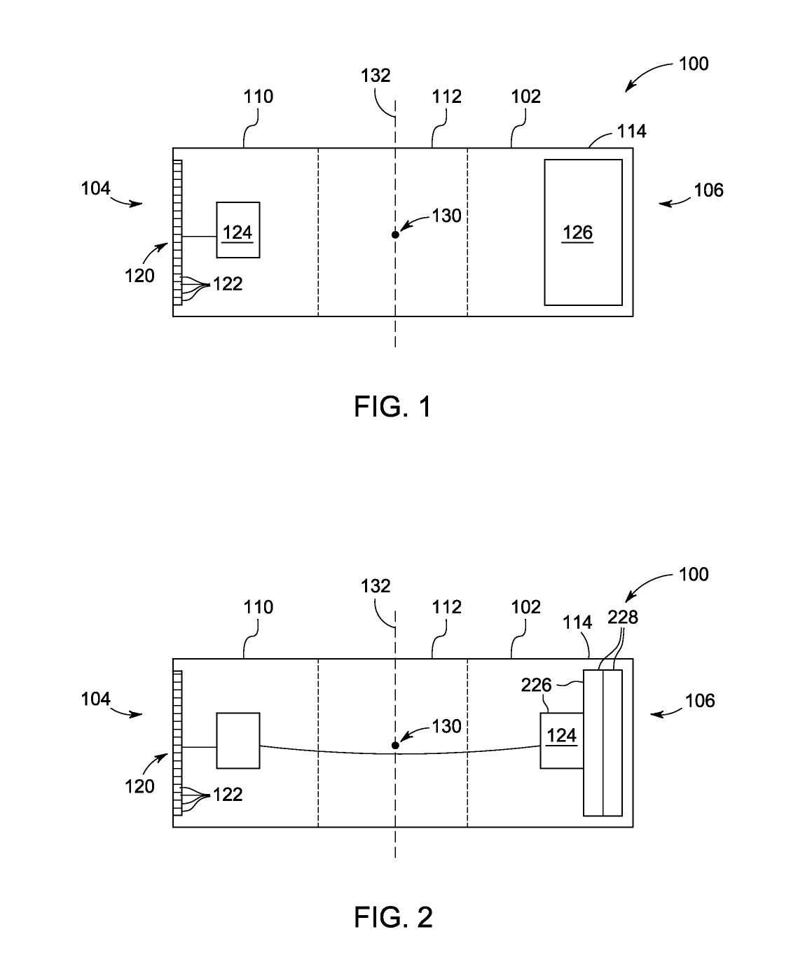

FIG. 1 illustrates a block diagram of an exemplary ultrasound probe 100 that is formed in accordance with various embodiments. The ultrasound probe 100 is configured to be secured to the palm of an operator's hand. In the exemplary embodiment, the ultrasound probe 100 enables the operator to position and control the operation of the ultrasound probe 100 without using the operator's fingers as is discussed in more detail below. Thus, the same hand of the operator may be used to perform concurrently both ultrasound imaging and other additional tasks, such as for example, operating and/or controlling other medical imaging equipment being used during the ultrasound imaging procedure.

The ultrasound probe 100 generally includes a housing 102 having a proximal end 104 and a distal end 106. The housing 102 includes a proximal portion 110, a connecting portion 112 and a distal portion 114. The proximal portion 110 is disposed proximate to the proximal end 104. The distal portion 114 is disposed proximate to the distal end 106. The connecting portion 112 is disposed between the proximal portion 110 and the distal portion 114. The proximal portion 110 generally includes therein control components and operating components for performing ultrasound scans. For example, and in general, the proximal portion 110 may include therein a transducer array 120 that is located at the proximal end 104 of the housing 102. The transducer array 120 may include a plurality of elements 122, for example, piezoelectric elements and control components 124, for example, electrical components mounted to a printed circuit board (not shown). The proximal portion 110 may be used to scan a patient by emitting therefrom ultrasonic waves and receiving echoes which are utilized to reconstruct an image of the area being scanned. It should be noted that the ultrasound probe 100 may include additional component parts, for example, a control knob (not shown) that is used to control the operation of the ultrasound probe. The connecting portion 112 couples the proximal portion 110 to the distal portion 114. Specifically, the connecting portion 112 provides a mating interface between the proximal portion 110 and the distal portion 114 to enable the proximal portion 110 to be physically coupled to the distal portion 114.

In the exemplary embodiment, the ultrasound probe 100 includes a counterweight 126 that is located within the distal portion 114 of the housing 102 proximate to the distal end 106. The counterweight 126 is configured to maintain a center of gravity 130 of the ultrasound probe 100 proximate to a centerline 132 of the housing 102. More specifically, the counterweight 126 is sized to define the center of gravity 130 of the ultrasound probe 100 proximate to the centerline 132 of the housing 102. For example, assuming that the components installed within the proximal portion 110, e.g. the transducer array 120 and the control components 124, have a total weight of W₁, a weight W₂ of the counterweight 126 is then selected to be approximately equal to W₁. Accordingly, in the exemplary embodiment W₁≈W₂. It should be realized that the weight of the counterweight 126 is selected based on the weight of the components installed in the proximal portion 110. Therefore, if the weight W₁ of the components in the proximal portion 110 is either increased or decreased, the weight W₂ of the counterweight 126 may be increased or decreased in proportion to the increase and/or decrease of the weight of the components in the proximal portion 110 to maintain the center of gravity 130 proximate to the probe centerline 132. The counterweight 126 may be formed as a separate component that is installed within the housing 102. Optionally, the counterweight 126 may be formed as an integral part of the housing 102.

For example, the housing 102 and the counterweight 126 may be formed as a single unitary component. Moreover, the housing 102 may be formed from two or more individual parts that are coupled together using, for example, an adhesive material or mechanical fasteners. The counterweight 126 may be formed from any material, e.g. a polymer, having a density that enables the material to be sized to include the predetermined weight W₂ and still be located within the ultrasound probe 100. In the exemplary embodiment, the counterweight 126 is installed within the housing 102 and the housing 102 is sealed to prevent bacterial and other undesirable substances from entering the ultrasound probe 100. Optionally, the distal portion 114, including the counterweight 126 may be fabricated as separate components that are coupled to the proximal portion 110 via the connecting portion 112.

The ultrasound probe 100, in one embodiment, is configured to generate ultrasound data based on the echoes and to wirelessly transmit the ultrasound data to a remote device that is configured to reconstruct an image based on the received data. Optionally, the ultrasound probe 100 may be hardwired to a remote device.

FIG. 2 illustrates a block diagram of another exemplary ultrasound probe 200 that is formed in accordance with various embodiments. The ultrasound probe 200 is also configured to be secured to the palm of an operator's hand. In the exemplary embodiment, the ultrasound probe 200 enables the operator to position and control the operation of the ultrasound probe 200 without using the operator's fingers. Thus, the operator may use the same hand to concurrently perform both ultrasound imaging and other additional tasks, for example, operating and/or controlling other medical imaging equipment being performed concurrently with the ultrasound imaging procedure.

The ultrasound probe 200, similar to ultrasound probe 100, generally includes the housing 102 having the proximal end 104 and the distal end 106. The housing 102 includes the proximal portion 110, the connecting portion 112 and the distal portion 114. The proximal portion 110 is disposed proximate to the proximal end 104. The distal portion 114 is disposed proximate to the distal end 106. The connecting portion 112 is disposed between the proximal portion 110 and the distal portion 114. As discussed above, in one embodiment, the control components 124 for operating the ultrasound probe 100 are located in the proximal portion 110 of the ultrasound probe 100. In this embodiment, the control components 124, or a portion of the control components 124, are located near the distal end 106 of the ultrasound probe 200, within the distal portion 114 and function as a counterweight 226.

The counterweight 226 is configured to maintain a center of gravity 130 of the ultrasound probe 200 proximate to the centerline 132 of the housing 102. As discussed above, the counterweight 226 should have a weight that is approximately equal to the weight of the components installed in the proximal portion 110. Therefore, a portion of the control components 124 may be installed in the proximal portion 110 and a second portion of the control components 124 may be installed in the distal portion 114. As discussed above, the total weight within the distal portion 114 should be substantially equal to the total weight of components within the proximal portion 110. Accordingly, some of the control components 124 may be located in proximal portion 110 and some of the control components 124 may be located in the distal portion 114. In one embodiment, if the control components 124 are installed in the distal portion 114 and the distal portion 114 remains lighter than the proximal portion 110, thus shifting the center of gravity 130 toward the proximal end 104, additional weights 228 may be installed within the distal portion 114 until the center of gravity 130 is proximate to the centerline 132 of the ultrasound probe 200. It should be realized that the center of gravity 130, in various embodiments, is located to enable the ultrasound probe 100 to be comfortably balanced in the operators hand.

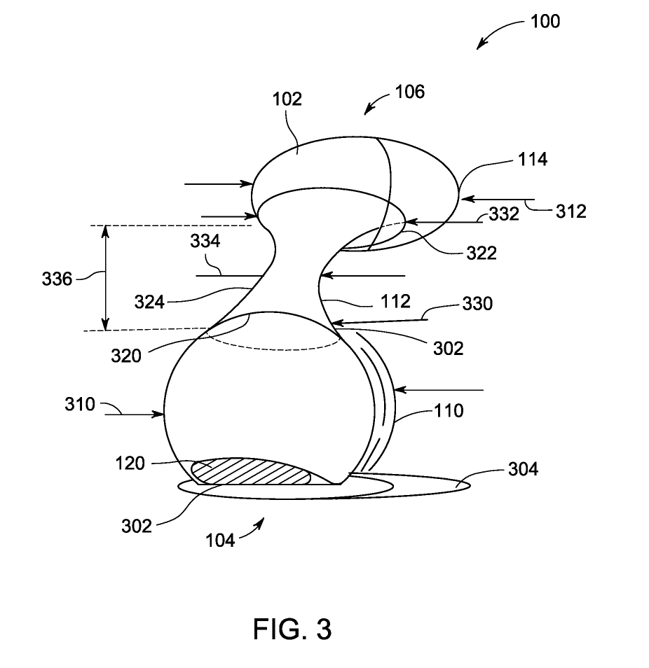

FIG. 3 is a perspective view of the ultrasound probes 100, 200 shown in FIGS. 1 and 2. Although the following description references the ultrasound probe 100 shown in FIG. 1, the description is also applicable to the ultrasound probe 200 shown in FIG. 2. As discussed above, the ultrasound probe 100 includes the proximal portion 110, the connecting portion 112, and the distal portion 114.

In the exemplary embodiment, the proximal portion 110 is formed to have a substantially spherical shape or oval shape that has a scanning surface 300 and an attachment surface 302. The scanning surface 300 is located at the proximal end 104 of the ultrasound probe 100. Preferably, the scanning surface 300 is formed to have a substantially planar surface to enable the transducer array 120, installed within the proximal portion 110, to be placed against a patients body 304. Optionally, the scanning surface 300 may be formed to have any shape that enables the transducer array 120 to be disposed on the patient 304 and a ultrasound scan to be performed.

The proximal portion 110 is formed to have a diameter 310. The diameter 310 is selected based on the size of the components desired to be installed within the proximal portion 110. More specifically, the diameter 310, or other dimensions, is selected based on the size of, for example, the transducer array 120 and any other control components 124 desired to be installed within the proximal portion 110. In the exemplary embodiment, the distal portion 114 may also be formed to have a substantially spherical shape. The distal portion 114 also has a diameter 312 that is selected based on the size of the components to be installed within the distal portion 114. More specifically, the diameter 312, or other dimensions, is selected based on the size of, for example, of any control components 124 and/or the counterweight 126 desired to be installed within the distal portion 114.

The connecting portion 112 is formed to have a first end 320, a second end 322, and a central portion 324. The first end 320 is coupled to the proximal portion 110 and the second end 322 is coupled to the distal portion 114. In the exemplary embodiment, the connecting portion 112 is formed as a unitary structure that is coupled between the proximal portion 110 and the distal portion 114. The connecting portion 112 has a length that is sufficient to enable the user to physically manipulate the probe 100. In some embodiments, the connecting portion 112 may be formed unitarily with the proximal portion 110 and the distal portion 114 to form a unitary housing 102. The first end 320 has a diameter 330, the second end 322 has a diameter 332, and the central portion 324 has a diameter 334. In one embodiment, the diameter 330 is substantially equal to the diameter 332, and the diameter 334 is less than the diameter 330 and 332.

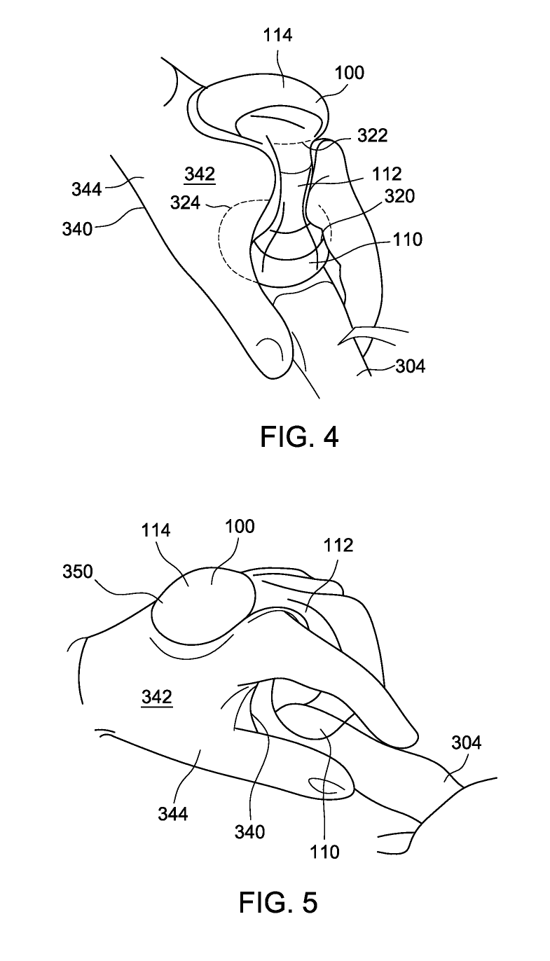

In another embodiment, the diameter 330 is different than the diameter 332, and the diameter 334 is less than the diameter 330 and 332. Forming the connecting portion 112 to have a narrower central portion enables the ultrasound probe 100 to be sized to be comfortably received between two of an operator's fingers. Moreover, a length 336 of the connecting portion 112 is selected to enable the proximal portion 110 to abut and/or rest against a palmar side 340 of an operators hand 342 and the distal portion 114 to abut and/or rest against a dorsal side 344 of the operators hand 342, as shown in FIG. 4. Accordingly, and referring again to FIG. 3, the connecting portion 112 tapers inwardly from the first end 320 to the center 324 and then tapers outwardly to the second end 322 to enable the ultrasound probe 100 to be friction fit to the hand 342.

Referring to FIGS. 4 and 5, in general, the ultrasound probe 100 is formed to have an ergonomic shape to enable the proximal portion 110 to rest comfortably against the palmar side 340 of the operators hand 342, and the distal portion 114 to rest against the dorsal side 344 of the operators hand 342. Thus, the exterior surface 350 of the ultrasound probe 100 is formed to have curves to enable the proximal portion 110 to smoothly transition to the connecting portion first end 320 and enable the connecting portion second end 322 to smoothly transition to the distal portion 114. Accordingly, in the exemplary embodiment, the exterior surface 350 of the ultrasound probe 100 is formed without any surfaces meeting at ninety degree angles, or angles proximate to ninety degrees, to provide a smooth exterior surface 350 that substantially conforms to the operator's hand 342 and eliminates any sharp edges or corners that may cause discomfort to the operator.

The proximal and distal portions 110 and 114 are preferably fabricated from an inelastic material that both protects the components installed within the ultrasound probe 100 and provides a smooth exterior surface 350 as discussed above. The inelastic material, in one embodiment, is a hard plastic material that is able to be sanitized for continuous and repeated operation. In one embodiment, the connecting portion 112 is fabricated from an elastic or resilient material that enables the connecting portion 112 to twist, bend, flex, or be otherwise deformed. More specifically, in operation, the operator may position the ultrasound probe 100 between the thumb and the index finger as shown in FIG. 4. The operator may also position the ultrasound probe 100 between the index finger and a middle finger as shown in FIG. 5. Optionally, the operator may position the ultrasound probe between any two fingers. The operator may then squeeze or otherwise manipulate the ultrasound probe 100 until the proximal portion 110 is contacting the palmar side 340 of the operators hand 342 and the distal portion 114 is contacting the dorsal side 344 of the operators hand 342. Once the ultrasound probe 100 is secured to the operator's hand, the operator may exert pressure on the connecting portion 112 to reposition the ultrasound probe 100 as needed to perform an ultrasound examination. Additionally, the connecting portion 112 enables the ultrasound probe 100 to remain coupled to the operator's hand without the use of the operator's fingers. Accordingly, the operator's fingers remain free to perform other tasks. For example, the operator may move or reposition the ultrasound probe 100 by exerting pressure against the connecting portion 112. Because the user's fingers are not required to manipulate the ultrasound probe 100, the user's fingers are free to perform other tasks, for example, holding the arm of a small child as shown in FIGS. 4 and 5. Moreover, because the connecting portion 112 is flexible, the ultrasound probe may be configured or deformed to fit to any size hand.

In operation, for example, the operator may desire to perform an ultrasound scan on a patient. The operator may then desire to temporarily interrupt the scanning procedure to perform another task, e.g. insert a catheter. In this case, because the ultrasound probe 100 is secured to the operator's hand without the use of the operator's fingers, the fingers of both hands remain free to perform the additional task without the operator having to remove the ultrasound probe 100 from his or her hand and place the ultrasound probe back onto a table, etc. After the ultrasound scan is completed, the operator may then exert force on any portion of the ultrasound probe 100 to remove the ultrasound probe 100 from his or her hand.

Accordingly, in this exemplary embodiment, the connecting portion 112 does not have a memory and the operator may reposition the proximal portion 110 with respect to the distal portion 114 to either attach or remove the ultrasound probe from the operator's hand. Additionally, the connecting portion 112 may be utilized to apply any amount of predetermined force to secure the ultrasound probe to the operator's hand. For example, the operator may apply a first force on the ultrasound probe 100 such that the ultrasound probe 100 is coupled tightly to the operator's hand. Optionally, the operator may apply a different second force on the ultrasound probe 100 such that the ultrasound probe 100 is coupled more loosely to the operator's hand. In all embodiments, the connecting portion 112 is configured to exert a predetermined force on the operator's hand. The force being sufficient to maintain the ultrasound probe 100 coupled to the user's hand. In this manner, the operator may manipulate the ultrasound probe 100 while the dorsal side 344 of the operator's hand is facing the ceiling, the floor, or in any other orientation without the ultrasound probe 100 becoming uncoupled from the operator's hand.

In another embodiment, the connecting portion 112 is resilient and has a memory. More specifically, the connecting portion 112 is formed such that the proximal portion 110 is separated from the distal portion 114 by a predetermined distance. The operator may then apply a force to the ultrasound probe 100 to separate the proximal portion 110 from the distal portion 114 while the ultrasound probe 100 is being installed on the operator's hand. The connecting portion 112 then attempts to return to its' predetermined shape and concurrently exerts a force that is sufficient to secure the ultrasound probe 100 to the operator's hand. After removing the ultrasound from the hand, the connecting portion 112 causes the proximal portion 110 and the distal portion 114 to return to the predetermined distance.

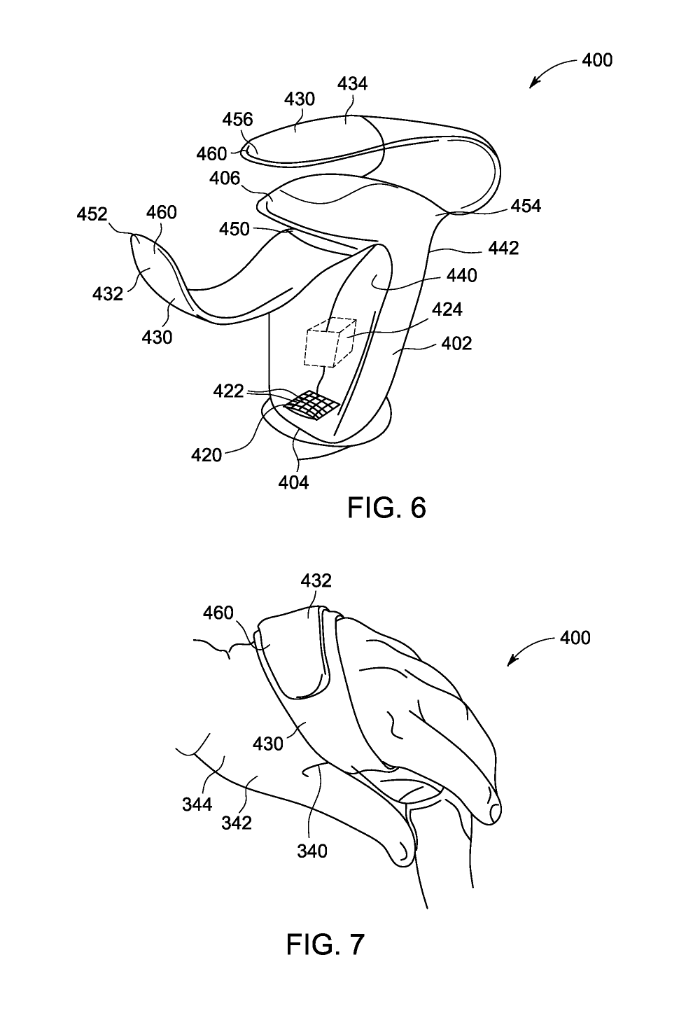

FIG. 6 is a perspective view of another ultrasound probe 400 formed in accordance with various embodiments. FIG. 7 is a perspective view of the ultrasound probe 400 shown in FIG. 6 in an exemplary operational position. The ultrasound probe 400 is configured to be secured to the palm of an operator's hand. In the exemplary embodiment, the ultrasound probe 400 enables the operator to position and control the operation of the ultrasound probe 400 without using the operator's fingers as is discussed in more detail below. Thus, the same hand of the operator may be used to perform concurrently both ultrasound imaging and other additional tasks, such as for example, operating and/or controlling other medical imaging equipment being performed concurrently with the ultrasound imaging procedure and/or holding the arm of the child while the ultrasound scan is being performed.

The ultrasound probe 400 generally includes a housing 402 having a proximal end 404 and a distal end 406. The distal end 406 is preferably shaped to have a curve that conforms to the hand 342. The ultrasound probe 400 generally includes therein control components and operating components for performing ultrasound scans. For example, and in general, the ultrasound probe 400 may include therein a transducer array 420 that is located at the proximal end 404 of the housing 402. The proximal end 404 may have a substantially flat or curved surface to enable the transducer array to contact the person being scanned. The transducer array 420 may include a plurality of elements 422, such as piezoelectric elements and control components 424, for example, electrical components mounted to a printed circuit board (not shown). It should be noted that the ultrasound probe 400 may include additional component parts, for example, a control knob (not shown) that is rotatable between an engaged and a disengaged position to control operation of the ultrasound probes.

A portion of the ultrasound probe 400 may be positioned on the dorsal side of the hand 342 and another portion may be positioned on the palmar side of the hand 342, similar to the ultrasound probes 100 and 200 discussed above. In this embodiment, the ultrasound probe 400 includes an elongated strap 430 that is configured to at least partially circumscribe the hand 342. In the illustrated embodiment, the strap 430 is positioned near the distal end 406 of the ultrasound probe 400. The strap 430 includes a first flexible strap portion 432 and a second flexible strap portion 434. The first strap portion 432 extends from a first side 440 of the ultrasound probe 400. The second strap portion 434 extends from an opposite second side 442 of the ultrasound probe 400. The first strap portion 432 has a first end 450 that is connected to or secured to the first side 440 of the ultrasound probe 400, between the distal end 406 and the proximal end 404. The first strap portion 432 also includes a second end 452 that is discussed in more detail below. The second strap portion 434 has a first end 454 that is connected to or secured to the second side 442 of the ultrasound probe 400, between the distal end 406 and the proximal end 404. The second strap portion 434 also includes a second end 456 that is discussed in more detail below. In one embodiment, the strap 430 is formed separately from the ultrasound probe 400 and then later secured to the ultrasound probe 400. Optionally, the strap 430 may be formed integrally with the ultrasound probe 400. The straps portions 432 and 434 are oriented such that the strap portions 432 and 434 extend from the distal end 406 of the ultrasound probe 400 and each wrap around the hand 342 is an opposite direction such that the ends of the strap portions 432 and 434 may be coupled together on the dorsal side of the hand 342 as shown in FIG. 6.

In the exemplary embodiment, the second ends 452 and 456 of the respective strap portions 432 and 434 function as a fastener 460 to secure the ultrasound probe 400 to the hand 342. The fastener 460 may be embodied, for example, as a hook and loop fastener. Optionally, the fastener 460 may be another suitable fastener type.

In the illustrated embodiment, the strap 430 extends from the palmar side 340 of the hand 342 to the dorsal side 344 of the hand 342. Moreover, the fastener 360 is located at an intermediate position on the dorsal side 344 of the hand 342 to enable the operator to operate the fastener 360 to secure the ultrasound probe 400 to the hand 342.

In one embodiment, the strap 430 is adjustable to enable the ultrasound probe 400 to be secured to any sized hand. For example, the strap 430 may be pulled less tight around the hand 342 to accommodate a larger size hand. Optionally, the strap 430 may be pulled tighter to achieve an opposite effect. The changing slack to tautness in the strap 430 can vary the force applied by the strap 430 to the hand 342 and thus vary the force utilized to secure the ultrasound probe 400 to the hand 342.

In use, to secure the ultrasound probe 400 to the hand 342, the first strap portion 432 is wrapped around the hand 342 in a first direction. The second strap portion 434 is then wrapped around the hand 342 in a second opposite direction. The ends 452 and 456 are then coupled together to secure the ultrasound probe 400 to the hand 342.

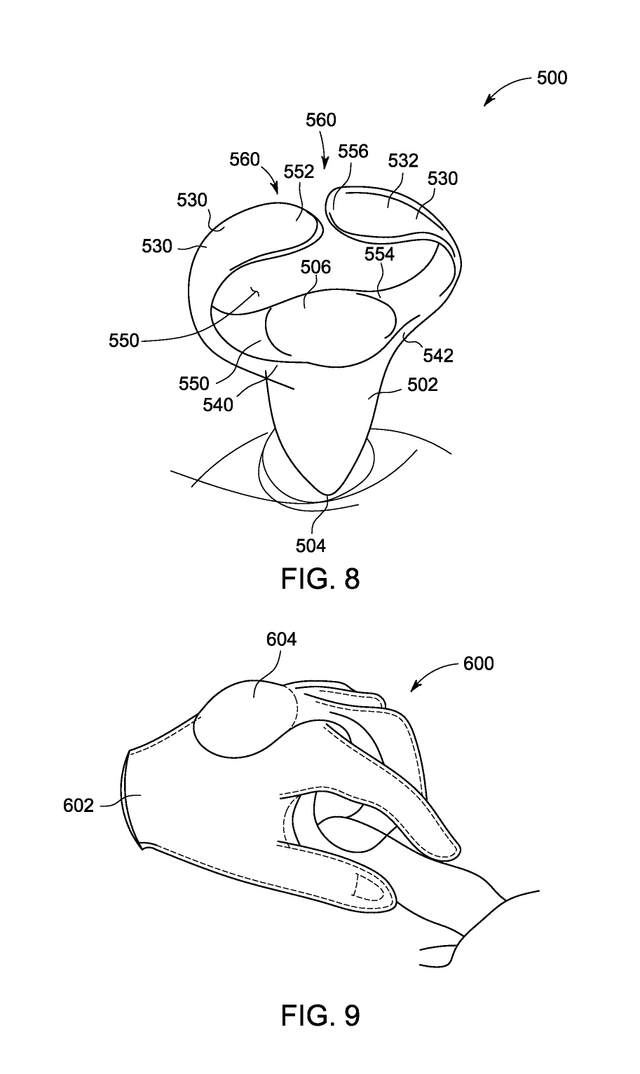

FIG. 8 is a perspective view of another ultrasound probe 500 formed in accordance with various embodiments. The ultrasound probe 500 is configured to be secured to the palm of an operator's hand. In the exemplary embodiment, the ultrasound probe 500 enables the operator to position, and control the operation, of the ultrasound probe 500 without using the operator's fingers. Thus, the same hand of the operator may be used to perform concurrently both ultrasound imaging and other additional tasks, for example, operating and/or controlling other medical imaging equipment or holding a child's are, concurrently with the ultrasound imaging procedure.

The ultrasound probe 500 generally includes a housing 502 having a proximal end 504 and a distal end 506. The distal end 506 is preferably shaped to have a curve that conforms to the hand 342 as shown in FIG. 4, for example. The ultrasound probe 500 generally includes therein control components and operating components for performing ultrasound scans. For example, and in general, the ultrasound probe 500 may include therein a transducer array (not shown) that is located at the proximal end 504 of the housing 502. The transducer array may include a plurality of elements (not shown), such as, for example, piezoelectric elements and control components (not shown), for example, electrical components mounted to a printed circuit board (not shown). It should be noted that the ultrasound probe 500 may include additional component parts, for example, a control knob (not shown) that is rotatable between an engaged and a disengaged position to control operation of the ultrasound probes.

A portion of the ultrasound probe 500 may be positioned on the dorsal side of the hand 342 and another portion of the ultrasound probe 500 may be positioned on the palmar side of the user's hand, similar to the ultrasound probes 100, 200, and 400 as discussed above. In this embodiment, the ultrasound probe 500 includes a strap 530. In the illustrated embodiment, the strap 530 is positioned near the distal end 506 of the ultrasound probe 500. The strap 530 includes a first flexible strap portion 532 and a second flexible strap portion 534. The first strap portion 532 extends from a first side 540 of the ultrasound probe 500. The second strap portion 534 extends from an opposite second side 542 of the ultrasound probe 500. The first strap portion 532 has a first end 550 that is connected to or secured to the first side 540 of the ultrasound probe 500. In this embodiment, the first strap portion 534, is connected to, and extends outwardly from the distal end 506. The first strap portion 532 also includes a second end 552 that is discussed in more detail below. The second strap portion 534 has a first end 554 that is connected to or secured to the second side 542 of the ultrasound probe 500. In this embodiment, the second strap portion 534, is connected to, and extends outwardly from the distal end 506. The second strap portion 534 also includes a second end 556 that is discussed in more detail below.

In one embodiment, the strap 530 is formed separately from the ultrasound probe 500 and then later secured to the ultrasound probe 500. In the exemplary embodiment shown in FIG. 8, the strap 530 is formed unitarily with the probe housing 502. Accordingly, the housing 502 and the strap 530 may be formed from the same material at the same time. In the exemplary embodiment, the second ends 552 and 556 of the respective strap portions 532 and 534 deform to secure the ultrasound probe 500 to the hand. More specifically, in this embodiment, the strap 530 may be formed from a semi-rigid material, e.g. plastic, that enables the strap 530 to temporarily deform while the operator is inserting his or her hand into the strap 530. The strap 530 then flexes back to its original shape to secure the ultrasound probe 500 to the user's hand.

The strap 530 is deformable to adjust to any sized hand. More specifically, the strap portions 532 and 534 may be fabricated from a material, such as plastic, that enables the strap portions 532 to be disposed or separated from each other by a predetermined distance when no force is exerted on the strap portions 532 and 534. The operator may then apply a force to the strap portions 532 and 534 to separate the strap portions 532 and 523 from each other and to create an opening 550 between the strap portions 532 and 534 that is sufficiently sized to enable the hand to be inserted through the opening 550. The strap portions 532 and 534 then return to the predetermined shape and concurrently exert a force that is sufficient to secure the ultrasound probe 500 to the operator's hand. After removing the ultrasound probe 500 from the hand, the strap portions 532 and 534 return to the original shape as when no force is being applied.

FIG. 9 is a perspective view of an exemplary ultrasound glove 600. The ultrasound glove 600 includes a glove portion 602 and an ultrasound probe 604. The ultrasound probe 604 may be embodied as any of the ultrasound probes describe herein or any other ultrasound probe. The ultrasound probe 604 is coupled to the glove portion 602 using an adhesive or a mechanical faster. In operation, the operator may simply insert a hand into the glove portion 602. Because the ultrasound probe 604 is secured to the glove portion 602, the ultrasound probe 604 remains securely coupled to the user's hand while the user is wearing the glove portion 602. Thus the other fingers in the glove portion 602 remain free to perform other tasks, for example, hold a child's arm while scanning the child's arm or utilizing a catheter, etc.

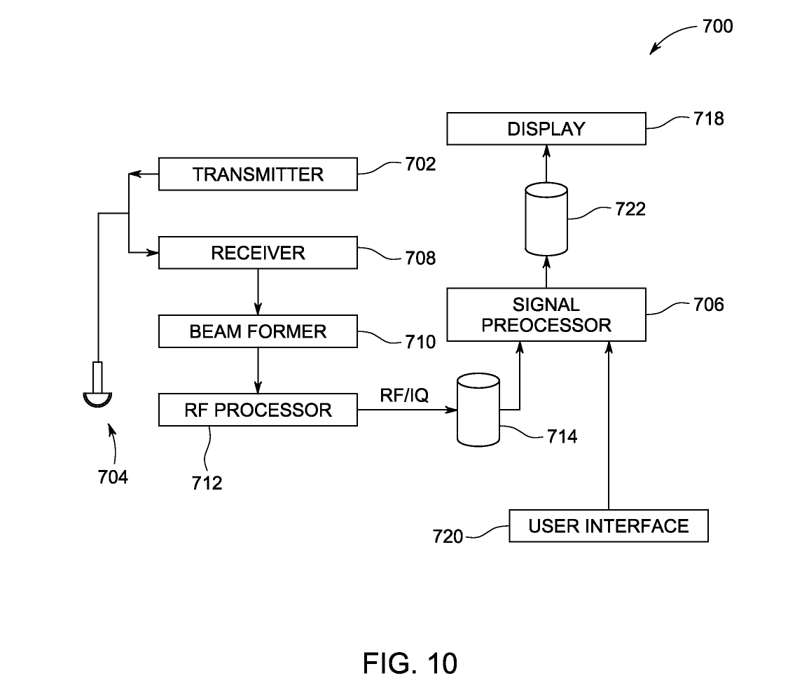

The various ultrasound probes described herein may be utilized with an imaging system such as the imaging system 700 shown in FIG. 10. Specifically, FIG. 10 illustrates a block diagram of an exemplary ultrasound system 700 that is formed in accordance with various embodiments. The ultrasound system 700 includes a transmitter 702 which operates an ultrasound probe, such as for example, the ultrasound probe 100. It should be realized that although the imaging system 700 is described with respect to ultrasound probe 100, any of the ultrasound probes 200, 400, 500, and/or 600 may also be utilized with the imaging system 700. In operation, the ultrasound probe 100 emits pulsed ultrasonic signals into an object or body. The ultrasound probe 100 may be used to acquire 2D, 3D, or 4D ultrasonic data, and may have further capabilities such as 3D beam steering. The ultrasonic signals are back-scattered from structures in the body, like blood cells or muscular tissue, to produce echoes which return to the ultrasound probe 100. The echoes are received by a receiver 708. The received echoes are passed through a beamformer 710, which performs beamforming and outputs an RF signal. The beamformer 710 may also process 2D, 3D and 4D ultrasonic data. The RF signal then passes through an RF processor 712. Alternatively, the RF processor 712 may include a complex demodulator (not shown) that demodulates the RF signal to form IQ data pairs representative of the echo signals. The RF or IQ signal data may then be routed directly to RF/IQ buffer 714 for temporary storage.

The ultrasound system 700 also includes a signal processor, such as signal processor 706. The signal processor 706 processes the acquired ultrasound information (i.e., RF signal data or IQ data pairs) and prepare frames of ultrasound information for display on a display 718. The signal processor 706 is adapted to perform one or more processing operations according to a plurality of selectable ultrasound modalities on the acquired ultrasound information. Acquired ultrasound information may be processed in real-time during a scanning session as the echo signals are received. Additionally or alternatively, the ultrasound information may be stored temporarily in the RF/IQ buffer 714 during a scanning session and processed in less than real-time in a live or off-line operation. A user interface, such as user interface 720, allows an operator to enter data, enter and change scanning parameters, access protocols, measure structures of interest, and the like. The user interface 720 may be a rotating knob, switch, keyboard keys, mouse, touch screen, light pen, or any other suitable interface device. Because the ultrasound probe 100 is secured to the hand, the operator may operate the user interface 720, various other components forming the imaging system 700, or other external components, without having to remove the ultrasound probe 100 from the hand.

The ultrasound system 700 may continuously acquire ultrasound information at a frame rate that exceeds 50 frames per second—the approximate perception rate of the human eye. The acquired ultrasound information, which may be the 3D volume dataset, is displayed on the display 718. The ultrasound information may be displayed as B-mode images, M-mode, volumes of data (3D), volumes of data over time (4D), or other desired representation. An image buffer 722 is included for storing processed frames of acquired ultrasound information that are not scheduled to be displayed immediately. Preferably, the image buffer 722 is of sufficient capacity to store at least several seconds worth of frames of ultrasound information. The frames of ultrasound information are stored in a manner to facilitate retrieval thereof according to its order or time of acquisition. The image buffer 722 may comprise any known data storage medium.

A technical effect of at least one embodiment is using the ultrasound probe concurrently while operating other devices. The various embodiments described herein facilitate enabling an operator to hold the ultrasound probe while enabling the operator to also operate other tools, such as a syringe or catheter, needed for an exam. The ultrasound probe is fixed on a part of the hand, allowing freedom for the fingers to manage any other accessory. The ultrasound probe performs the scan while the operator may also perform other activities concurrently.

In various embodiments, the ultrasound probe communicates wirelessly with a remote imaging system. The compactness and the shape of the ultrasound probe permits the ultrasound probe to be positioned on the hand in an orientation to perform scanning while also allowing the fingers to move freely. Accordingly, the fingers are not utilized to hold the ultrasound probe while the scanning procedure is performed. In various embodiments, the connecting portion compresses the ultrasound probe in between the palm and the scanned patient body. The ultrasound probe may not be fixedly coupled to the operator's hand such that the ultrasound probe is unable to move with respect to the hand. Rather, the ultrasound probe is configured to be held or rest on the hand. Various embodiments reduce examination time and reduce procedural complexity by enabling the operator to utilize both hands.

Exemplary embodiments of an ultrasound probe and an ultrasound system are described above in detail. The ultrasound probe and system components illustrated are not limited to the specific embodiments described herein, but rather, components of each ultrasound system may be utilized independently and separately from other components described herein. For example, the ultrasound probes described above may also be used in combination with other imaging systems.

The computer or processor executes a set of instructions that are stored in one or more storage elements, in order to process input data. The storage elements may also store data or other information as desired or needed. The storage element may be in the form of an information source or a physical memory element within a processing machine.

The set of instructions may include various commands that instruct the computer or processor as a processing machine to perform specific operations such as the methods and processes of the various embodiments of the invention. The set of instructions may be in the form of a software program. The software may be in various forms such as system software or application software. Further, the software may be in the form of a collection of separate programs, a program module within a larger program or a portion of a program module. The software also may include modular programming in the form of object-oriented programming. The processing of input data by the processing machine may be in response to user commands, or in response to results of previous processing, or in response to a request made by another processing machine.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. While the dimensions and types of materials described herein are intended to define the parameters of the invention, they are by no means limiting and are exemplary embodiments. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. §112, sixth paragraph, unless and until such claim limitations expressly use the phrase “means for” followed by a statement of function void of further structure.

What is claimed is:

1. An ultrasound probe comprising:
a proximal portion including a transducer array;
a distal portion; and
a connecting portion configured to couple the scanning portion to the distal portion, the connecting portion sized to be received between two fingers to enable the ultrasound probe to be secured to an operator's palm.

2. The ultrasound probe of claim 1, wherein the proximal portion is configured to abut a palmar side of an operator's hand and the distal portion is configured to abut a dorsal side of the operator's hand.

3. The ultrasound probe of claim 1, wherein the connecting portion is configured to enable an operator to reposition the ultrasound probe without the use of an operator's fingers.

4. The ultrasound probe of claim 1, wherein the distal portion comprises a counterweight that creates a center of gravity proximate to a centerline of the ultrasound probe.

5. The ultrasound probe of claim 1, wherein the distal portion comprises a control section coupled to the transducer array, the control section configured to operate the transducer array and function as a counterweight that creates a center of gravity proximate to a centerline of the ultrasound probe.

6. The ultrasound probe of claim 1, wherein the distal portion comprises a counterweight having a first weight and the proximal portion has a second weight that is substantially equal to the first weight.

7. The ultrasound probe of claim 1, wherein the connecting portion is formed from a deformable material to enable the ultrasound probe to elastically deform to an operator's hand.

8. The ultrasound probe of claim 1, wherein the connecting portion is deformable to enable the ultrasound probe to be friction fit to the operator's palm.

9. The ultrasound probe of claim 1, wherein the connecting portion is sized to be received between an index finger and a second finger or the index finger and a thumb.

10. The ultrasound probe of claim 1, wherein the distal end has a curved shape to conform to the operator's palm.

11. The ultrasound probe of claim 1, wherein the proximal portion and the distal portion comprise a first material and the connecting portion comprises a different second material.

12. The ultrasound probe of claim 1, wherein the connecting portion gradually tapers from a center of the connecting portion outwardly to both the proximal portion and the distal portion.

13. An ultrasound probe comprising:
a housing having a proximal end and a distal end, the distal end being curved to conform to an operator' palm;
a transducer array located within the housing; and
a strap coupled to the housing, the strap configured to at least partially circumscribe the hand and to secure the ultrasound probe to the operator's palm.

14. The ultrasound probe of claim 13, wherein the strap comprises:
a first strap portion; and
a second strap portion, the first and second strap portions being formed unitarily with the housing.

15. The ultrasound probe of claim 13, wherein the strap comprises:
a first strap portion; and
a second strap portion, the first and second strap portions having a predetermined shape, the first and second strap portions configured to deform to define an opening that is sized to receive a hand therethrough.

16. The ultrasound probe of claim 13, wherein the strap comprises:
a first strap portion; and
a second strap portion, the second strap portion configured to couple to the first strap portion using a fastening system.

17. An ultrasound imaging system comprising:
an ultrasound probe comprising:
a proximal portion including a transducer array;
a distal portion; and
a connecting portion configured to couple the scanning portion to the distal portion, the connecting portion sized to be received between two fingers to enable the ultrasound probe to be secured to an operator's palm; and
a processor communicatively coupled with the ultrasound probe, the processor receiving ultrasound data from the ultrasound probe and generating at least one image based on the ultrasound data.

18. The ultrasound system of claim 17, wherein the proximal portion is configured to abut a palmar side of an operator's hand and the distal portion is configured to abut a dorsal side of the operator's hand.

19. The ultrasound system of claim 17, wherein the connecting portion is configured to enable an operator to reposition the ultrasound probe without the use of an operator's fingers.

20. The ultrasound system of claim 17, wherein the distal portion comprises a counterweight that creates a center of gravity proximate to a centerline of the ultrasound probe.
